SEQUENCE LISTING (7 sequences)

SEQ ID NO: 1 - DNA, 879 bp, Artificial Sequence
  DNS-Fragment xI
  misc_feature (1)..(879)
caattcgcgcgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaagaattctaaggaggaaattatatggtcaaatctcaaccgattttgagatatatcttgcgcgggattcccgcgattgcagtagcggttctgctttctgcatgtagtgcaaataacaccgcaaagaatatgcatcctgagacacgtgcagtgggtagtgaaacatcatcactgcaagcttctcaggatgaatttgaaaacctggttcgtaatgtcgacgtaaaatcgcgaattatggatcagtatgctgactggaaaggcgtacgttatcgtctgggcggcagcactaaaaaaggtatcgattgttctggtttcgtacagcgtacattccgtgagcaatttggcttagaacttccgcgttcgacttacgaacagcaggaaatgggtaaatctgtttcccgcagtaatttgcgtacgggtgatttagttctgttccgtgccggttcaacgggacgccatgtcggtatttatatcggcaacaatcagtttgtccatgcttccaccagcagtggtgttattatttccagcatgaatgaaccgtactggaagaagcgttacaacgaagcacgccgggttctcagccgcagctaagaattgcaagctggccgacgcgtcccacagccgccagttccgctggcggcattttaactttctttaatgaagccggaaaaatcccgcgcgcgaaggc

SEQ ID NO: 2 - DNA, 1131 bp, Artificial Sequence
  DNS-Fragment xII
  misc_feature (1)..(1131)
caattcgcgcgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaagaattctaaggaggaaattatatggcgcggataaaccgtatttcgatcacgctctgtgctttactttttaccaccctgcctttaacgcctatggcccatgcttcaaagcaagccagggagagttctgctaccactcatatcaccaaaaaagcagataaaaagaaaagcacggcaaccaccaaaaagacccagaaaacagcgaaaaaagccgccagtaaaagtacgaccaaaagcaaaaccgcttcttccgttaaaaaatcttccattaccgcttctaaaaacgccaaaactcgcagcaaacacgccgtcaataaaacggcctcagccagctttaccgaaaagtgtaccaaacgtaagggctataaatcgcattgtgtgaaagtcaaaaatgccgcgtcaggaactcttgccgacgcgcacaaagcgaaggtgcaaaaagctacgaaagtggcaatgaataaactgatgcagcaaattggtaagccatatcgttggggtggcagctcaccgcgtaccggttttgattgcagcggcctggtttattacgcttataaagatttggtgaaaattcgtattccgcgtacggcgaatgaaatgtatcacctgcgtgatgcagcgccaatcgaacgtagtgaactgaaaaacggcgacctggtctttttccgtactcagggacgcggcacagccgatcatgtcggcgtgtatgtcggcaacggcaaatttattcagtcaccgcgcacaggtcaggaaattcaaatcacttctctcagtgaagactactggcagcgccactatgttggcgctcgtcgggtaatgaccccaaaaacacttcgctaagaattgcaagctggccgacgcgctctcccacagccgccagttccgctggcggcattttaactttctttaatgaagccggaaaaatcccgcgcgcgaaggc

SEQ ID NO: 3 - DNA, 1599 bp, Artificial Sequence
  DNS-Fragment xIII
  misc_feature (1)..(1599)
acagaattctaaggaggaaattatatgaaaagaaaccgtttttttaatacctcggctgctattgccatttcgattgcattacagatcttttttccgtccgcttccgctttcgctcaggttcagctggtgcagagcggtgccgaagttgttaaaccgggtgcaagcgttaaactgagctgtaaagcaagcggctatatttttaccagctattatatgtattgggtgaaacaggcaccgggtcagggtctggaatggattggtgaaattaatccgagcaatggcgataccaattttaatgaaaaatttaaaagcaaagccaccctgaccgttgataaaagcgcaagcaccgcatatatggaactgagcagcctgcgtagcgaagataccgcagtttattattgtacccgtagtgatggtcgcaatgatatggatagctggggtcagggcaccctggtcaccgttagcagcgcaagcaccaaaggtccgagcgtttttccgctggcaccgagcagcaaaagcaccagcggtggcaccgcagcactgggttgtctggttaaagattattttccggaaccggttacagttagctggaatagcggtgcactgaccagtggtgttcatacctttccggcagttctgcaaagcagcggtctgtatagcctgagcagcgttgttaccgttccgagcagtagcctgggcacccagacctatatttgtaatgttaatcataaaccgagcaacaccaaagtggataaaaaagttgaaccgaaaagctgctaataaccatggagaaaataaagtgaaacaaagcactattgcactggcactcttaccgttactcttcacccctgttaccaaagccgatattgtgctcacccagagtccggcaaccctgagcgttagtccgggtgaacgtgcaaccattagctgtcgtgcaagccagcgtgttagcagcagcacctatagctatatgcattggtatcagcagaaaccgggtcagcctccgaaactgctgattaaatatgcaagcaatctggaaagcggtgttccggcacgttttagcggtagcggtagtggcaccgattttaccctgaccattagcagcgttgaaccggaagattttgccacctattattgtcagcatagctgggaaattcctccgacctttggtggtggcaccaaactcgagattaaacgtaccgttgcagcaccgagcgtgtttatctttccgcctagtgatgaacagctgaaaagcggcaccgcaagcgttgtttgtctgctgaataacttttatccgcgtgaagcaaaagttcagtggaaagttgataatgcactgcaaagcggtaatagccaagaaagcgttaccgaacaggatagcaaagatagcacctatagcctgtcaagcaccctgaccctgagcaaagcagattatgaaaaacacaaagtgtatgcctgcgaagttacccatcagggtctgagcagtccggttacaaaaagttttaatcgtggtgaatgtagctcttctgcccatcaccaccatcaccattaataagcttctagaagcttggctgttttggcggatgagagaagattttcgac

SEQ ID NO: 4 - DNA, 564 bp, Escherichia coli
  misc_feature (1)..(564): coding for the Spr precursor protein
atggtcaaatctcaaccgattttgagatatatcttgcgcgggattcccgcgattgcagtagcggttctgctttctgcatgtagtgcaaataacaccgcaaagaatatgcatcctgagacacgtgcagtgggtagtgaaacatcatcactgcaagcttctcaggatgaatttgaaaacctggttcgtaatgtcgacgtaaaatcgcgaattatggatcagtatgctgactggaaaggcgtacgttatcgtctgggcggcagcactaaaaaaggtatcgattgttctggtttcgtacagcgtacattccgtgagcaatttggcttagaacttccgcgttcgacttacgaacagcaggaaatgggtaaatctgtttcccgcagtaatttgcgtacgggtgatttagttctgttccgtgccggttcaacgggacgccatgtcggtatttatatcggcaacaatcagtttgtccatgcttccaccagcagtggtgttattatttccagcatgaatgaaccgtactggaagaagcgttacaacgaagcacgccgggttctcagccgcagc

SEQ ID NO: 5 - protein, 188 aa, Escherichia coli
  PEPTIDE (1)..(188): amino acid sequence of Spr precursor protein
MVKSQPILRYILRGIPAIAVAVLLSACSANNTAKNMHPETRAVGSETSSLQASQDEFENLVRNVDVKSRIMDQYADWKGVRYRLGGSTKKGIDCSGFVQRTFREQFGLELPRSTYEQQEMGKSVSRSNLRTGDLVLFRAGSTGRHVGIYIGNNQFVHASTSSGVIISSMNEPYWKKRYNEARRVLSRS

SEQ ID NO: 6 - DNA, 813 bp, Escherichia coli
  misc_feature (1)..(813): DNA coding for the Ydho precursor protein
atggcgcggataaaccgtatttcgatcacgctctgtgctttactttttaccaccctgcctttaacgcctatggcccatgcttcaaagcaagccagggagagttctgctaccactcatatcaccaaaaaagcagataaaaagaaaagcacggcaaccaccaaaaagacccagaaaacagcgaaaaaagccgccagtaaaagtacgaccaaaagcaaaaccgcttcttccgttaaaaaatcttccattaccgcttctaaaaacgccaaaactcgcagcaaacacgccgtcaataaaacggcctcagccagctttaccgaaaagtgtaccaaacgtaagggctataaatcgcattgtgtgaaagtcaaaaatgccgcgtcaggaactcttgccgacgcgcacaaagcgaaggtgcaaaaagctacgaaagtggcaatgaataaactgatgcagcaaattggtaagccatatcgttggggtggcagctcaccgcgtaccggttttgattgcagcggcctggtttattacgcttataaagatttggtgaaaattcgtattccgcgtacggcgaatgaaatgtatcacctgcgtgatgcagcgccaatcgaacgtagtgaactgaaaaacggcgacctggtctttttccgtactcagggacgcggcacagccgatcatgtcggcgtgtatgtcggcaacggcaaatttattcagtcaccgcgcacaggtcaggaaattcaaatcacttctctcagtgaagactactggcagcgccactatgttggcgctcgtcgggtaatgaccccaaaaacacttcgc

SEQ ID NO: 7 - protein, 271 aa, Escherichia coli
  PEPTIDE (1)..(271): Amino acid sequence of the Ydho precursor protein
MARINRISITLCALLFTTLPLTPMAHASKQARESSATTHITKKADKKKSTATTKKTQKTAKKAASKSTTKSKTASSVKKSSITASKNAKTRSKHAVNKTASASFTEKCTKRKGYKSHCVKVKNAASGTLADAHKAKVQKATKVAMNKLMQQIGKPYRWGGSSPRTGFDCSGLVYYAYKDLVKIRIPRTANEMYHLRDAAPIERSELKNGDLVFFRTQGRGTADHVGVYVGNGKFIQSPRTGQEIQITSLSEDYWQRHYVGARRVMTPKTLR

(19) United States
(12) Patent Application Publication
(10) Pub. No.: US 2024/0327849 A1
(43) Pub. Date: Oct. 3, 2024

(54) BACTERIAL STRAIN FOR RELEASING A RECOMBINANT PROTEIN IN A FERMENTATION METHOD

(71) Applicant: Wacker Chemie AG, Munich (DE)

(72) Inventors: Johanna Koch, München (DE); Marian Kujau, Jena (DE)

(73) Assignee: Wacker Chemie AG, Munich (DE)

(21) Appl. No.: 17/632,936

(22) PCT Filed: Aug. 5, 2019
(86) PCT No.: PCT/EP2019/071006

§ 371 Date: Jun. 6, 2024

(51) Int. Cl. C12N 15/70; C07K 16/28; C12N 9/10; C12N 9/48

(52) U.S. Cl. CPC C12N 15/70; C07K 16/2875; C12N 9/1074; C12N 9/48; C12Y 204/01019; C07K 2317/14; C07K 2317/55; C07K 2319/02

(57) ABSTRACT

The invention relates to a bacterial strain containing an open reading frame encoding a signal peptide and a recombinant protein under the control of a functional promoter. The bacteria strain contains an additional open reading frame encoding for a signal peptide and a peptidoglycan peptidase under the control of a functional promoter.

The invention relates to a bacterial strain containing an open reading frame encoding a signal peptide and a recombinant protein under the control of a functional promoter, characterized in that the bacterial strain contains an additional open reading frame encoding a signal peptide and a peptidoglycan peptidase under the control of a functional promoter. The invention further relates to a plasmid encoding a recombinant protein and a peptidoglycan peptidase and to a method for fermentative production of recombinant proteins using the bacterial strain according to the invention.

Recombinant proteins can be produced cost-effectively in bacteria because of their short generation time and the simple handling in comparison with mammalian cell cultures. Owing to its extensively studied genetics and physiology, the Gram-negative enterobacterium Escherichia coli is currently the most commonly used organism for production of recombinant proteins. What are particularly attractive are production methods for recombinant proteins in E. coli in which the target protein is released directly into the fermentation medium in high yield and in the correct folding, since this avoids complicated cell-disruption and protein-folding processes.

A range of E. coli strains that achieve release of recombinant proteins into the culture medium are disclosed in the literature. These are mutants of E. coli that have a permanent defect in the outer membrane and therefore partially discharge periplasmic proteins into the culture medium. This is a nonspecific mechanism. An example of such “leaky” mutants are strains having defects in the Tol-Pal complex, for example tol- and pal-deletion mutants. It is known that tol- and pal-deletion mutants discharge cell-endogenous periplasmic proteins into the culture medium. Such strains are extremely sensitive to EDTA, various detergents and antibiotics and exhibit growth defects (Clavel et al. 1998, Mol. Microbiol. 29, pages 359-367: Chen et al. 2014, Microb. Biotechnol. 7, pages 360-370). Therefore, they are of only limited suitability for culturing at high cell density and over a relatively long period.

For this reason, preference is given to using strains which can release proteins into the culture medium without having permanent defects. The inducible release of recombinant proteins at a particular time point in a production process can be achieved by weakening of the peptidoglycan layer, which is an essential stability factor with regard to the bacterial cell envelope.

The peptidoglycan or murein layer is a crosslinked macromolecule which occurs in the cell envelope of Gram-positive and Gram-negative bacteria and gives it strength. The peptidoglycan consists of strands of interlinked molecules of N-acetylglucosamine and N-acetylmuramic acid that form the backbone as a linear chain. The N-acetylmuramic acid residues of adjacent strands are linked to one another via an oligopeptide (Silhavy et al. 2010, Cold Spring Harb. Perspect. Biol. 2(5)).

Peptidoglycan hydrolases are a group of enzymes which can cleave covalent bonds in the peptidoglycan layer (Vollmer, FEMS Microbiol Rev. 32, 2008, pages 259-286). The peptidoglycan hydrolases are divided into different classes depending on the type of bond that is cleaved. Glycosidases (N-acetylglucosaminidases, lytic transglycosylases), which also include lysozymes, cleave the glycosidic bonds of the peptidoglycan backbone made up of N-acetylmuramic acid and N-acetylglucosamine units. Amidases (N-acetylmuramoyl-L-alanine amidases) cleave the amide bond between the N-acetylmuramic acid of the peptidoglycan backbone and the L-alanine of the peptide cross-link. Peptidases (carboxypeptidases and endopeptidases) by contrast cleave the peptide bonds within the peptide cross-link.

A known example of the use of a peptidoglycan hydrolase in the prior art is the addition of lysozyme to cells following harvesting of a bacterial culture in order to disrupt said cells (Pierce et al. 1997, J Biotechnol 58, pages 1-11).

For instance, in the patent document CN 104762285 B, ClyN, a fusion of lysozyme and peptidoglycan binding domains, is produced in an inducible manner and cytoplasmic green fluorescent protein (GFP) that has been produced recombinantly is thus released from the cells into the culture medium.

A further known principle is the co-transformation of E. coli with a vector for production of a recombinant protein and a vector for production of lysozyme from the T4 phage to release the recombinant protein such as, for example, cytoplasmic β-glucuronidase (Morita et al. 2001, Biotechnol. Prog. 17, pages 573-576) or an anti-CD18 antibody fragment or IGF-I (EP 1 124 941).

The production of lysozyme leads to the lysis of the cells, meaning that these approaches have the major disadvantage that the release of the recombinant proteins is accompanied by the release of other intracellular constituents, such as host cell proteins and DNA. This contaminates the culture supernatant, the consequence of this being work-up difficulties, especially because of an increased viscosity. This disadvantageous effect is described in CN 104762285 B for the release of GFP by ClyN, in which the live cell count after induction of ClyN production falls to 0.2% compared to a culture without induction of ClyN production. Owing to the cell lysis, the optical density of the E. coli culture expressing ClyN drops by approx. two thirds of the value before the start of ClyN production within 30 min. After 5 h, the optical density of the culture expressing ClyN is only approx. 20% of the optical density of a comparative culture with no ClyN production. The release of β-glucuronidase due to T4-phage lysozyme in Morita et al. is associated with a significantly lower optical density of the culture.

The same disadvantageous effect is reported in EP 1 124 941 for the release of the anti-CD18 antibody fragment, where the overproduction of the T4-phage lysozyme led to a decrease in cell density to 30% of the maximum value. In the case of the release of IGF-I due to T4-phage lysozyme, a sharp decrease in cell density due to lysis was likewise observed, and microscopic analysis revealed only a few intact cells.

It is an object of the present invention to provide a bacterial strain which, compared to a wild-type host cell, releases a recombinant protein into the culture medium in an increased yield without the occurrence of strong cell lysis.

This object is achieved by a bacterial strain containing an open reading frame encoding a signal peptide and a recombinant protein under the control of a functional promoter, characterized in that the bacterial strain contains an additional open reading frame encoding a signal peptide and a peptidoglycan peptidase under the control of a functional promoter.

The bacterial strain is preferably characterized in that the bacterial strain is Gram-negative bacteria, particularly preferably a bacterial strain of the genus Enterobacteriaceae, especially preferably a strain of the species Escherichia coli.

The wild-type gene refers to the form of the gene that arose naturally by evolution and is present in the wild-type bacterial genome.

The gene expresses the precursor protein, which comprises the amino acid sequence of the entire protein including the amino acid sequence for the signal sequence and does not bear post-translational modifications.

The term mature protein refers to the protein which comprises the amino acid sequence of the entire protein with the signal sequence removed and in which any post-translational modifications are present. Such a post-translational modification is, for example, modification with a membrane anchor.

In the context of this invention, the term full-length protein refers to the mature protein.

According to the invention, the additional open reading frame contained in the bacterial strain encodes a signal peptide and a peptidoglycan peptidase that is one of the peptidoglycan hydrolases. In what follows, the term “peptidoglycan peptidase” always means the additional protein expressed by said additional ORF in its mature form, i.e., after the signal peptide has been cleaved off, and not the protein encoded by the gene which is present in the bacterial wild-type genome and is expressed. Examples of genes encoding peptidoglycan peptidases in E. coli are spr, ydhO, yebA, dacA, dacC, dacD, yfeW, pbpG, mepA, dacB, ampH and ldcA.
1. In a preferred embodiment, the peptidoglycan peptidase encoded by the additional ORF is a wild-type full-length protein which cleaves the bond between the D-alanine and the meso-diaminopimelic acid of the peptide side chain, such as Spr (Uniprot entry POAFV4), YdhO (Uniprot entry P76190), YebA (Uniprot entry P0AFS9), MepA (Uniprot entry P0C0T5), DacB (Uniprot entry P24228) or PBP 7 (Uniprot entry P0AFI5), having the amino acid sequence known from the specified database entries,
2. in an alternatively preferred embodiment, the peptidoglycan peptidase encoded by the additional ORF is a mutated form of a wild-type full-length protein having a sequence identity of at least 30%, preferably at least 70%, to the amino acid sequence of the corresponding wild-type full-length protein,
3. in a further preferred embodiment, the peptidoglycan peptidase encoded by the additional ORF is a fragment of a wild-type full-length protein,
4. in a further preferred embodiment, the peptidoglycan peptidase encoded by the additional ORF is a mutated version of a fragment of a wild-type protein having a sequence identity of at least 30%, preferably at least 70%, to the amino acid sequence of the corresponding fragment of a wild-type full-length protein, wherein, in all cases 1-4, the protein encoded by the additional ORF and chosen as the peptidoglycan peptidase has peptidoglycan peptidase activity. Methods as to how this activity can be detected are known to a person skilled in the art from the literature (Gonzalez-Leiza et al. 2011, J Bacteriol 193, pages 6887-94).

The mutations at the amino acid level, as described, for example, under point 2 and point 4, can be substitutions, deletions and/or insertions compared to the wild-type full-length protein of peptidoglycan peptidase or fragments thereof.

Examples of peptidoglycan peptidases are Spr and YdhO, which cleave the bond between D-alanine and meso-diaminopimelic acid in the peptide cross-links of the bacterial peptidoglycan layer (Singh et al. 2012, Mol. Microbiol. 86, pages 1036-1051) and thus allow the incorporation of new strands into the existing peptidoglycan network and hence cell growth.

The spr gene from E. coli encodes a Spr protein having 188 amino acids (Uniprot POAFV4), with amino acids 1-26 forming the signal sequence for transport from the cytosol into the periplasm. In the mature Spr protein, the signal sequence is proteolytically cleaved off and, in addition, the cysteine residue at position 1 of the mature protein (position 27 of the total sequence) is modified with a membrane anchor, via which the Spr protein is anchored in the outer membrane. The mature Spr protein accordingly consists of 162 amino acids and is referred to in the context of this invention as Spr full-length protein.

The ydhO gene encodes a YdhO protein having 271 amino acids (Uniprot P76190). Amino acids 1-27 are predicted to be the periplasmic export signal peptide. The mature YdhO protein accordingly consists of 244 amino acids and is referred to in the context of this invention as YdhO full-length protein.

The DNA sequence encoding the Spr precursor protein is specified in SEQ ID No. 4, and the DNA sequence encoding the YdhO precursor protein is specified in SEQ ID No. 6. The amino acid sequence of the Spr precursor protein is specified in SEQ ID No. 5, with amino acids 1-26 forming the signal sequence and amino acids 27-188 forming the mature Spr protein (to be equated with the Spr full-length protein). The amino acid sequence of the YdhO precursor protein is specified in SEQ ID No. 7, with amino acids 1-27 forming the signal sequence and amino acids 28-271 forming the mature YdhO protein (to be equated with the YdhO full-length protein).

Preferably, the peptidoglycan peptidase is (i) Spr, (ii) YdhO or (iii) Spr and YdhO Said Spr and YdhO is the wild-type full-length protein respectively specified in the database sequence, a mutated form of the wild-type full-length protein having a sequence identity of at least 30%, preferably at least 70%, to the amino acid sequence of the wild-type full-length protein, a fragment of the wild-type full-length protein, or a mutated version of a fragment of the wild-type full-length protein having a sequence identity of at least 30%, preferably at least 70%, to the amino acid sequence of a fragment of the wild-type full-length protein, wherein the protein chosen has murein DD-endopeptidase activity. Especially preferably, Spr is the sequence specified by amino acids 27-188 in SEQ ID No. 5 and YdhO is the sequence specified by amino acids 28-271 in SEQ ID No. 7.

In the preferred embodiment in which the peptidoglycan peptidase is Spr, it is particularly preferred that the Spr protein interacts with the outer membrane.

The DNA sequences encoding the peptidoglycan peptidase according to the invention are preferably a DNA sequence which originates from Escherichia coli.

Open reading frame (ORF) refers to that region of the DNA or RNA that is between a start codon and a stop codon and encodes the amino acid sequence of a protein. The ORF is also referred to as a coding region.

ORFs are surrounded by noncoding regions. Gene refers to the DNA segment which contains all the basic information for producing a biologically active RNA. A gene contains the DNA segment from which a single-stranded RNA copy is produced by transcription and the expression signals which are involved in the regulation of this copying process. The expression signals include, for example, at least a promoter, a transcription start site, a translation start site and a ribosome binding site. Furthermore, a terminator and one or more operators are possible as expression signals.

In the case of a functional promoter, the ORF which is under the regulation of said promoter is transcribed into an RNA.

Monocistronic refers to a messenger RNA (mRNA) which contains only one open reading frame.

An operon is a functional unit of DNA that comprises multiple ORFs, a promoter and possibly further expression signals.

The bacterial strain can contain (1) one ORF encoding a signal peptide and a recombinant protein, (2) multiple ORFs encoding the same signal peptide and recombinant protein or (3) multiple ORFs encoding a different signal peptide and recombinant protein in each case. Whereas the 2nd possibility is, for example, used for increasing the expression and yield of the recombinant protein, the 3rd possibility is used especially in the expression of proteins which are composed of multiple polypeptides (subunits). One example is the expression of antibody fragments which are subsequently assembled after transport out of the cytoplasm.

Each of these ORFs can be in a separate gene: it is also possible for multiple ORFs to be organized in an operon, i.e., to be regulated by common expression signals.

If the recombinant protein is composed of multiple polypeptide chains (subunits), it is preferred that the ORFs of the individual peptide chains (subunits), each linked to a signal peptide, is organized in an operon.

Each operon can moreover contain one ORF encoding a signal peptide and a peptidoglycan peptidase.

It is preferred that the ORF encoding a signal peptide and the peptidoglycan peptidase is transcribed as monocistronic messenger RNA. This means that the peptidoglycan peptidase is expressed by a separate gene of its own. In the preferred embodiment in which the peptidoglycan peptidase is Spr or YdhO, the corresponding gene is referred to as the spr gene or ydhO gene.

The ORF(s) encoding the signal peptide(s) and recombinant protein(s) and the signal peptide(s) and peptidoglycan peptidase(s) can be expressed chromosomally or by a plasmid. In the case of separate genes, they can also be expressed by separate plasmids compatible with one another with regard to origin of replication and selection marker. The ORFs are preferably plasmid-encoded.

The origin of the DNA sequence of the ORF according to the invention encoding a peptidoglycan peptidase is not restricted to the bacterial strain used for production of the recombinant protein, so long as the corresponding peptidoglycan peptidase is functional in the bacterial strain used for production of the recombinant protein, i.e., has murein DD-endopeptidase activity (see above). Preferably, the sequence of the ORF encoding the peptidoglycan peptidase protein is a DNA sequence which originates from the same organism also used for production of the recombinant proteins.

The term “a/the ORF encoding a signal peptide and a recombinant protein” and “a/the recombinant protein”, which is used in the singular in the context of this invention, can also mean multiple ORFs and/or multiple different recombinant proteins, each led by a signal peptide. Preferably 1 to 3 different recombinant proteins, particularly preferably I recombinant protein or 2 different recombinant proteins, are concerned. The cloning and expression of recombinant proteins in bacteria is achieved as described in the prior art. The recombinant protein has, for example, a signal sequence for transport into the periplasm.

The recombinant protein is a protein which the wild-type bacterial genome naturally either does not express at all or expresses in a different amount. The bacterium serves for the production of the recombinant protein, said protein being, according to the invention, released into the culture medium.

The use of the bacterial strain according to the invention in a fermentation method has the advantage that said bacterial strain expresses a peptidoglycan peptidase as an additional protein together with the recombinant protein. The term “additional” refers to the fact that a further or “additional” open reading frame encoding a peptidoglycan peptidase is present in addition to the wild-type gene in the bacterial strain. This additional open reading frame encoding a peptidoglycan peptidase is preferably located on a plasmid.

Owing to its signal sequence, the peptidoglycan peptidase is localized in the periplasm and leads to destabilization of the peptidoglycan layer as a result of the cleavage of the peptide cross-links. Despite the destabilization of the cell envelope of the bacterium, no increased cell lysis was observed and, in the experiments, the cell dry weight of the bacterial strain expressing a recombinant protein and the peptidoglycan peptidase remained the same compared to the bacterial strain expressing only the recombinant protein. As a result of the destabilization of the cell envelope of the bacterium, recombinant proteins can be released from the periplasm of the cell and can be isolated from the culture supernatant in increased yield. In this way, it is possible to achieve increased product yields in the culture supernatant compared to the prior art without the occurrence of substantial death of the bacterial cells.

Increased yield is to be understood to mean that, using the bacterial strain according to the invention, preferably at least 110%, particularly preferably at least 150% and especially preferably at least 200% of the amount of recombinant protein is released into the culture medium, compared to what can be produced according to the prior art using a wild-type bacterial strain containing the same gene for a recombinant protein. This means that the yield of recombinant protein which is released into the culture medium is preferably at least 1.1 times, particularly preferably at least 1.5 times and especially preferably at least 2 times as high as the yield which can be achieved with corresponding bacterial strains having no overproduction of a peptidoglycan peptidase.

In the present invention, CGTase was used as an example of a recombinant protein. The comparison of CGTase yield in Example 1 shows that the yield of CGTase measured in U/ml when using the W3110/pCGT-Spr bacterial strain is increased by almost 10-fold compared to the W3110/pCGT bacterial strain (see Table 1). Neither the cell dry weight (CDW) nor the live cell count was reduced compared to the control strain as a result of the additional production of the Spr protein. The strain W3110/pCGT-YdhO achieved almost the same CGTase yield as the strain W3110/pCGT-Spr (see Table 2) without substantially influencing the cell dry weight compared to the control batch containing the W3110/pCGT bacterial strain.

For the production of a Fab antibody fragment as recombinant protein, in this case the CD154 Fab fragment, Example 2 also confirms that a substantially higher yield of recombinant protein could be achieved using the strain W3110/pJF118ut-CD154-Spr expressing the peptidoglycan peptidase Spr compared to the strain W3110/pJF118ut-CD154, in this case a good 200-times higher (Table 3). At the same time, the cell dry weight decreased only slightly (Table 4).

OD₆₀₀ refers to the optical density of the cell culture determined with the aid of a spectrophotometer at 600 nm and is a measure of the quantity of cells per unit of volume (cell concentration, cell density), which is in turn a measure of cell division activity and a measure of cell lysis, with cell lysis leading to a decrease in cell density.

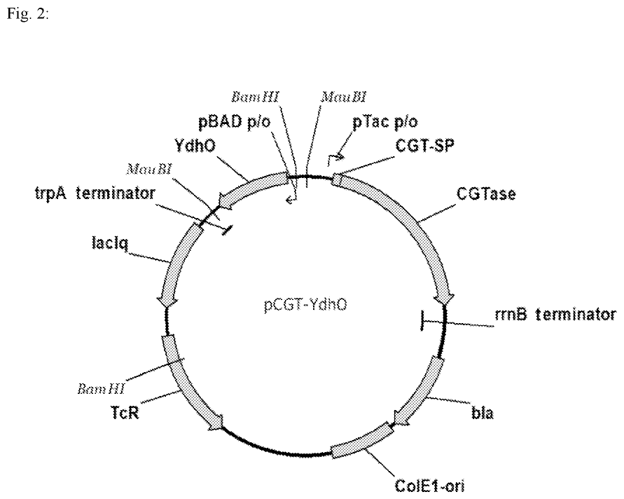

CN 104762285 B shows by contrast in FIG. 2 that the OD600 value, i.e., cell density, falls substantially after induction of the expression of ClyN, which implies strong cell lysis.

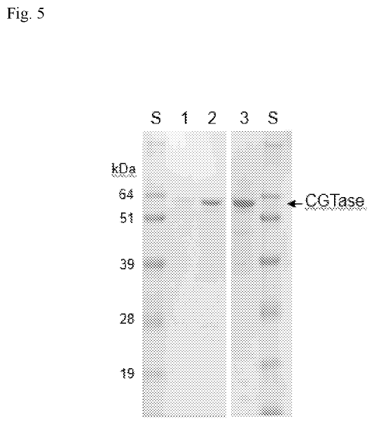

Since strong cell lysis leads to the release of host cell proteins, the recombinant protein becomes contaminated with host cell proteins. In addition, the release of DNA leads to an increase in viscosity in the culture medium, which makes further work-up more difficult. Since the bacterial strain according to the invention does not exhibit an increased rate of cell lysis, use of said strain for production of recombinant proteins means that the recombinant protein does not become contaminated with host cell proteins. This is confirmed by the analysis of the protein content of the culture supernatant by means of SDS-PAGE in Example 1 (FIG. 5), which shows that severe contamination of the recombinant protein by host cell proteins did not occur when the bacterial strain according to the invention was used. The additionally expressed peptidoglycan peptidase did not exhibit increased release either.

To achieve the release of the recombinant protein into the culture medium, it is necessary for both the recombinant protein and the precursor peptidoglycan peptidase to be transported into the periplasm after protein biosynthesis in the cytosol. For transport into the periplasm, it is necessary to link the 5′ end of the coding DNA sequence of the recombinant protein and the 5′ end of the coding DNA sequence of the peptidoglycan peptidase in frame with the 3′ end of a signal sequence for protein export. Suitable for this purpose are, in principle, all signal sequences which allow translocation of the target protein with the aid of the Sec or Tat apparatus in the bacterial strain used. Various signal sequences are described in the prior art, for example the signal sequences of the following genes: phoA, ompA, pelB, ompF, ompT, lamB, malE, Staphylococcal protein A, StII and others (Choi and Lee 2004, Appl. Microbiol. Biotechnol. 64, pages 625-635). What is preferred according to the invention for recombinant proteins is the signal sequence of the phoA gene or the ompA gene of E. coli or the signal sequence for a cyclodextrin glycosyltransferase (α-CGTase) from Klebsiella pneumoniae M5a1 or signal sequences derived therefrom that are disclosed in US 2008/0076157 as SEQ ID No. 1 and 3. The peptidoglycan peptidase preferably bears in its precursor form the signal sequence native to the protein in question. In the preferred embodiment in which the peptidoglycan peptidase is Spr or YdhO, the precursor form of the peptidoglycan peptidase in question is the sequence specified in SEQ ID No. 5 and 7, respectively. The recombinant protein and the peptidoglycan peptidase preferably bear a differing signal sequence, the two signal sequences bringing about protein export.

The expression of the ORFs encoding the peptidoglycan peptidase and the recombinant protein can be controlled by one promoter, by two identical promoters or by two different promoters. It is preferred that the ORFs for the peptidoglycan peptidase and for the recombinant protein are under the control of two different promoters.

The DNA segment encoding the peptidoglycan peptidase can be first amplified by means of PCR using oligonucleotides as primers and a DNA template encoding the peptidoglycan peptidase, for example genomic DNA isolated from E. coli, and then, using common molecular-biology techniques, linked in each case with the DNA molecule comprising the sequence of a signal peptide and generated in an analogous manner, such that an in-frame fusion is formed. Alternatively, it is also possible to produce the entire DNA molecule by means of gene synthesis. Said DNA molecule consisting of the signal sequence in question and the coding sequence for the mature protein can then either be introduced into a vector, for example a plasmid, or be directly integrated by known methods into the chromosome of the bacterial strain. Preferably, the DNA molecule is introduced into a plasmid, such as, for instance, a derivative of known expression vectors, such as pJF118EH, pKK223-3, pUC18, pBR322, pACYC184, pASK-IBA3 or pET. Plasmids are introduced into the bacterial cells using methods known by a person skilled in the art (transformation).

The plasmids used can bear selection markers. Suitable as selection markers are genes which encode a resistance to antibiotics such as, for example, ampicillin, tetracycline, chloramphenicol, kanamycin or others. Preferably, the plasmid contains a gene, the expression of which mediates tetracycline resistance. Suitable as selection markers are, furthermore, auxotrophic markers which encode an essential gene which has been deleted in the bacterial strain in question that contains the plasmid. If two plasmids are transformed into the cells, preference is given to selecting for the presence of both plasmids using two different antibiotic resistances, one antibiotic resistance and one auxotrophic marker or two different auxotrophic markers.

Suitable as promoters are all promoters known to a person skilled in the art, such as constitutive promoters such as, for example, the GAPDH promoter or inducible promoters such as, for example, the lac, tac, trc, lambda PL, ara, cumate or tet promoter or sequences derived therefrom. The ORFs encoding the recombinant protein and the ORF encoding the peptidoglycan peptidase can be controlled as an operon by a promoter or be under the control of different promoters. Preferably, the functional promoter which controls the expression of the open reading frame encoding the peptidoglycan peptidase is an inducible promoter.

Preferably, the ORFs encoding the recombinant protein and the ORF encoding the peptidoglycan peptidase are controlled by different inducible promoters. Particularly preferably, the recombinant protein is expressed under the control of the tac promoter and the peptidoglycan peptidase is expressed under the control of the ara (arabinose) promoter.

Preferably, the recombinant protein is a heterologous protein. Heterologous proteins are to be understood to mean proteins which do not belong to the proteome, i.e., the entire natural set of proteins, of the bacterial strain, preferably an E. coli K12 strain. All proteins occurring naturally in the bacterial strain used, for example in the E. coli K12 strain, can be derived from the known genome sequences (e.g., from the Genbank entry under accession No. NC_000913 for E. coli K12).

Particularly preferred as heterologous proteins are eukaryotic proteins which contain one or more disulfide bonds. Especially preferred are eukaryotic proteins which, in their functional form, are present as dimers or multimers.

The most important heterologous protein classes include antibodies and fragments thereof, cytokines, growth factors, protein kinases, protein hormones, lipocalins, anticalins, enzymes, binding proteins and molecular scaffolds and proteins derived therefrom. Examples of said protein classes are, inter alia, heavy-chain antibodies and fragments thereof (e.g., nanobodies), single-chain antibodies, interferons, interleukins, interleukin receptors, interleukin receptor antagonists, G-CSF, GM-CSF. M-CSF, leukemia inhibitors, stem cell growth factors, tumor necrosis factors, growth hormones, insulin-like growth factors, fibroblast growth factors, platelet-derived growth factors, transforming growth factors, hepatocyte growth factors, bone morphogenetic factors, nerve growth factors, brain-derived neurotrophic factors (BDNF), glial cell line-derived neurotrophic factors, angiogenesis inhibitors, tissue plasminogen activators, blood coagulation factors, trypsin inhibitors, elastase inhibitors, complement constituents, hypoxia-induced stress proteins, proto-oncogenic products, transcription factors, virus-constitutive proteins, proinsulin, prourokinase, erythropoietin, thrombopoietin, neurotrophin, protein C, glucocerebrosidase, superoxide dismutase, renin, lysozyme, P450, prochymosin, lipocortin, reptin, serum albumin, streptokinase, tenecteplase, CNTF and cyclodextrin glycosyltransferases.

Examples of proteins derived from molecular scaffolds are, inter alia, evibodies (derived from CTLA-4), affibodies (protein A of S. aureus), avimers (of human A domain family), transbodies (of transferrin), DARPins (of ankyrin repeat protein), adnectin (of fibronectin III), peptide aptamers (of thioredoxin), microbodies (of microprotein), affilins (of ubiquitin), α-crystallin, charybdotoxin, tetranectin, PDZ domain of the RAS-binding protein AF-6, Kunitz-type domain of protein inhibitors.

In the context of this invention, a selection marker that is possibly used is not considered to be a gene encoding a recombinant protein. The term selection marker refers to a gene which is introduced into the modified organism as a marker together with the “gene of interest”, i.e., the gene which is actually desired and encodes the recombinant protein, in order to be able to identify individuals having a successful gene modification. Commonly used selection markers are antibiotic resistances or auxotrophies. The marker genes provide a cell or an organism with a survival advantage under certain conditions, an example being that the expression of β-lactamase by the ampicillin resistance gene as a marker gene of the cell allows survival in ampicillin-containing medium. By contrast, recombinant proteins are produced with the aid of the bacterial strain and isolated after a certain culture period.

The invention further provides a plasmid containing an open reading frame encoding a signal peptide and a recombinant protein under the control of a functional promoter, characterized in that said plasmid additionally contains an open reading frame encoding a signal peptide and a peptidoglycan peptidase under the control of a functional promoter.

The preferred and particularly preferred features mentioned for the bacterial strain according to the invention also apply to the features mentioned in the plasmid according to the invention, wherein the preferred and particularly preferred embodiments mentioned for the bacterial strain according to the invention are also respectively preferred or particularly preferred in the plasmid. All of the preferred features mentioned for the “additional” ORF present in the bacterial strain and encoding a signal peptide and a peptidoglycan peptidase and mentioned for the gene encoding the recombinant protein also apply to this ORF encoding a signal peptide and a peptidoglycan peptidase and the gene encoding the recombinant protein.

Similarly, the mentioned definitions and preferred embodiments apply to the ORF encoding a signal peptide and a peptidoglycan peptidase and to the ORF encoding the signal peptide and the recombinant protein.

For example, one or more ORFs encoding one or more signal peptides and recombinant proteins can be in one operon. Preferably, the ORF encoding the signal peptide and the peptidoglycan peptidase is in a separate gene. This means that the peptidoglycan peptidase and the recombinant protein can be expressed independently of one another. In the particularly preferred case of the promoter(s) of the recombinant protein(s) being different from the promoter of the peptidoglycan peptidase and differently inducible, the plasmid has the particular advantage that, upon transformation of bacteria with said plasmid, it is possible to choose the optimal time point for the expression of the proteins independently of one another.

Preferably, the plasmid is characterized in that the DNA encoding the peptidoglycan peptidase is a gene selected from the group of the genes spr and ydhO. The definition given above applies. In this preferred embodiment, the DNA sequence of the peptidoglycan peptidase is SEQ ID No. 4 and/or 6, i.e., the plasmid encodes the Spr and/or YdhO precursor protein, i.e., the plasmid encodes the amino acid sequence of the respective mature protein and the respective signal peptide. Particularly preferably, the plasmid is characterized in that the peptidoglycan peptidase is SEQ ID No. 4. As an alternative preference, the plasmid is characterized in that the peptidoglycan peptidase is SEQ ID No. 6.

Preferably, the plasmid is characterized in that the functional promoter which controls the expression of the open reading frame encoding a signal peptide and the peptidoglycan peptidase is an inducible promoter.

For examples of inducible promoters and for preferred embodiments, what has been said above applies.

The introduction of the plasmid according to the invention into the bacterial strain using methods known to a person skilled in the art and the expression of the recombinant protein and of the peptidoglycan peptidase by said plasmid has the advantage that the release of the recombinant proteins from the cells of the bacterial strain is improved and that they can be isolated in an increased yield.

In comparison with chromosomal integration of the ORFs encoding the recombinant protein and the peptidoglycan peptidase, the advantage of the use of plasmids is that the plasmid-bearing cells of the bacterial strain have a selection advantage and can be selected by standard methods.

Furthermore, it is of particular advantage that plasmids can be present in high copy number in the cells of the bacterial strain.

The invention further provides a method for fermentative production of recombinant proteins, characterized in that a bacterial strain according to the invention is cultured in a fermentation medium, the fermentation medium is removed from the cells after the fermentation, and recombinant proteins are isolated from the fermentation medium.

The cells of the bacterial strain that have been transformed by chromosomal integration or with one or two expression plasmids are cultured by customary methods known to a person skilled in the art in a shake flask or in a bioreactor (fermenter).

Possibilities as fermentation media (growth media, culture media) are, in principle, all common media known to a person skilled in the art for culturing bacteria. In this connection, it is possible to use complex media or minimal salts media to which a particular proportion of complex components, such as, for example, peptone, tryptone, yeast extract, molasses or corn steep liquor, is added. Furthermore, it is possible to add to the medium further components, such as vitamins, salts, amino acids and trace elements, which improve cell growth.

The fermentation is preferably carried out in a conventional bioreactor, for example, a stirred tank, a bubble column fermenter or an airlift fermenter. Particular preference is given to a stirred tank fermenter.

The fermentation involves culturing the cells of the protein production strain in a growth medium with ongoing monitoring and precise control of various parameters, such as, for example, nutrient feed, oxygen partial pressure, pH and temperature of the culture. The culturing period is preferably 24-65 h.

The primary carbon source used for the fermentation can, in principle, be all sugars, sugar alcohols or organic acids or salts thereof that are utilizable by the cells. In this connection, preference is given to using glucose, lactose, arabinose or glycerol. Particular preference is given to glucose and arabinose. Also possible is a combined feeding of multiple different carbon sources. At the same time, the carbon source can be initially charged in full in the fermentation medium at the start of fermentation, or nothing or only a portion of the carbon source is initially charged at the start and the carbon source is fed over the course of the fermentation. Particularly preferred in this connection is one embodiment in which a portion of the carbon source is initially charged and a portion is fed. Particularly preferably, the carbon source glucose is initially charged in a concentration of 10-30 g/l, and the feeding is started when the concentration has dropped to less than 5 g/l in the course of the fermentation and is configured such that the concentration is held below 5 g/l.

If the expression of the recombinant protein and/or of the peptidoglycan peptidase is controlled by an inducible promoter, the expression is induced by addition of the promoter-corresponding inducer to the fermentation batch. Suitable as inducers are, for example, arabinose, lactose, IPTG, tetracycline or cumate. The inducer can be metered in at any desired time point during the fermentation as a single or multiple dose. Alternatively, the inducer can be added continuously. Preferably, the expression of the recombinant protein is induced by addition of IPTG and the expression of the peptidoglycan peptidase is induced by addition of arabinose. Particularly preferably, IPTG is metered in as a single dose. The induction of the expression of the peptidoglycan peptidase is preferably configured such that a mixture of glucose and arabinose is fed at a time point after the glucose concentration has dropped below 5 g/l, or the arabinose is metered in as a single dose.

The expression of the peptidoglycan peptidase is preferably induced in the culturing phase in which there are only a few to no more cell divisions, i.e., just before or at the start of the stationary phase of the growth curve. This time point is determined by the cell density exhibiting only a little rise or no longer exhibiting a rise at all, which cell density is determined as OD₆₀₀ value or CDW value (see below).

Preferably, the medium in the fermenter is stirred before inoculation and sparged with sterilized compressed air: in this connection, the oxygen content is calibrated to 100% saturation and a target value for the O₂ saturation during the fermentation is chosen, which is between 10% and 70%, preferably between 20% and 60% and particularly preferably at 30% of this value. After the O₂ saturation has fallen below the target value, a regulation cascade starts in order to bring the O₂ saturation back to the target value, it being possible to adjust gas supply and stirring speed.

The pH of the culture is preferably between pH 6 and pH 8. Preferably, a pH between 6.5 and 7.5 is set: particularly preferably, the pH of the culture is held between 6.8 and 7.2.

The temperature of the culture is preferably between 15° C. and 45° C. Preference is given to a temperature range between 20° C. and 40° C., particular preference is given to a temperature range between 25° C. and 35° C., and very particular preference is given to the temperature of 30° C.

According to the invention, the fermentation medium is removed from the cells after the fermentation, and recombinant proteins are isolated from the fermentation medium. This can be done by customary methods, as are known in the prior art. Customarily, the cells are, in a first step, removed from the recombinant proteins released into the culture medium by means of separation methods such as centrifugation or filtration. The recombinant proteins can then, for example, be concentrated by ultrafiltration.

The expression of the peptidoglycan peptidase improves the release of recombinant proteins into the culture supernatant. As a result of the improved release, recombinant proteins can be isolated in an increased yield.

For the term “increased yield”, the above definition applies.

With the aid of the coexpression of the peptidoglycan peptidase in a bacterial strain, preferably in E. coli, it is also possible to produce Fab antibody fragments extracellularly. In this case, the bacterial cell must simultaneously synthesize the corresponding fragments of the light chain, which comprises the domains VL and CL, and of the heavy chain, which comprises the domains VH and CHI, of the antibody and then secrete them into the periplasm. Outside the cytoplasm, the two chains then assemble to form the functional Fab fragment. The corresponding ORFs can be in different genes. It is preferred that the ORFs encoding antibody fragments of the light and heavy chain are organized in an operon. As a result of simultaneous production of the peptidoglycan peptidase according to the invention, the heavy and the light chain of the antibody are released into the fermentation medium in an increased concentration.

The method has the major advantage that it is suitable for producing recombinant proteins which require a long culture period and for fermentation at high cell densities of >40 g/l. As a result, it is possible to produce complex, eukaryotic proteins as recombinant proteins.

Preferably, the method is characterized in that the recombinant proteins are purified from the fermentation medium after the removal of the fermentation medium.

Recombinant proteins can be further purified via standard methods such as precipitation or chromatography. Particular preference is given here to methods such as affinity chromatography, which utilizes the already correctly folded native conformation of the protein.

Preferably, the method is characterized in that the expression of the peptidoglycan peptidase is induced. This means that either only the expression of the peptidoglycan peptidase or the expression of the recombinant protein and the expression of the peptidoglycan peptidase is induced. This means that, in this preferred embodiment, the expression of the peptidoglycan peptidase is in any case under the control of an inducible promoter. By contrast, the expression of the recombinant protein can be under the control of a constitutive or an inducible promoter. Preferably, both the ORF encoding the signal peptide and the recombinant protein and the ORF encoding the signal peptide and the peptidoglycan peptidase are under the control of an inducible promoter, particularly preferably of differently inducible promoters. Therefore, the expression of the peptidoglycan peptidase and that of the recombinant protein is preferably inducible.

Since the promoters of the genes encoding the recombinant protein and the peptidoglycan peptidase can preferably be induced independently of one another, it is possible to choose independently of one another the optimal time point for the expression of the recombinant protein and for the expression of the peptidoglycan peptidase.

By using inducible promoters, it is possible to induce the expression of the corresponding proteins at any desired time point of the fermentation. Preferably, the expression of the peptidoglycan peptidase is induced after the induction of the expression of the recombinant protein. Particularly preferably, the expression of the peptidoglycan peptidase is induced at least 1 hour, especially preferably at least 2 hours, further particularly preferably 15 to 40 hours and specifically preferably 20, 27 or 38 hours after the induction of the expression of the recombinant protein.

The induction of the expression of the recombinant protein and of the peptidoglycan peptidase is triggered by addition of the inducer to the culture medium, it being necessary to choose the inducer in line with the gene used. In the preferred embodiment in which the peptidoglycan peptidase is under the control of the arabinose (ara) promoter, the expression of the peptidoglycan peptidase is induced by addition of the inducer arabinose to the culture. In the preferred embodiment in which the recombinant gene contains a tac promoter, the expression of the recombinant protein is induced by addition of the inducer lactose or of the lactose analog IPTG to the culture.

According to the invention, even after induction of the expression of the peptidoglycan peptidase and further culture of the cells, the cell density of the strain is comparable to a bacterial strain which does not express an additional peptidoglycan peptidase. In particular, a strong decrease in cell dry weight due to cell lysis does not occur.

The invention has the advantage that recombinant proteins are released into the culture medium without the occurrence of strong lysis of the cells of the bacterial strain. The method according to the invention is therefore distinguished by being suitable both for producing complex, eukaryotic proteins having a correspondingly long culture period and for fermentation at high cell densities >40 g/l.

A long culture period means that the culturing time is at least 24 h, preferably at least 48 h and particularly preferably at least 65 h.

In the context of the invention, a relatively low (or only slightly increased or non-strong) cell lysis means that there is only a little to absolutely no reduction in the CDW value, OD₆₀₀ value or the value for the live cell count of the culture of the bacterial strain that is determined in the case of a culture period of preferably 7-17 h after induction of the expression of the peptidoglycan peptidase in comparison with the wild-type bacterial strain which does not produce an additional peptidoglycan peptidase. The bacterial strains expressing a peptidoglycan peptidase have a great advantage especially for the culture of bacterial strains expressing peptidoglycan glycosidases whose cell lysis is strongly increased, as shown by Morita et al. (2001, see above) and in the patent documents CN 104762285 and EP 1 124 941 for cells expressing T4-phage lysozyme or the ClyN protein.

Preferably, the method is characterized in that, 5 to 24 hours after induction of the expression of the additional peptidoglycan peptidase, the cell dry weight differs by not more than 20% from the cell dry weight of a cell culture at the same time point from a fermentation method for a bacterial strain which does not express the additional peptidoglycan peptidase. Preferably, a cell culture from the fermentation method according to the invention is characterized in that, 5 to 24 hours, particularly preferably 7 hours or 17 hours, after induction of the expression of the additional peptidoglycan peptidase, it has a cell dry weight that is lower by not more than 20%, particularly preferably not more than 15% and especially preferably not more than 10%, than the cell dry weight of a cell culture at the same time point from a fermentation method which only differs from the method according to the invention in that a bacterial strain is cultured which only differs from the bacterial strain according to the invention in that it does not contain the ORF encoding a peptidoglycan peptidase. Cell dry weight serves as a measure of cell density and hence of non-lysed cells. Cell dry weight is determined by isolating a defined quantity of culture by filtration or centrifugation, preferably centrifugation, and subsequently drying and weighing it.

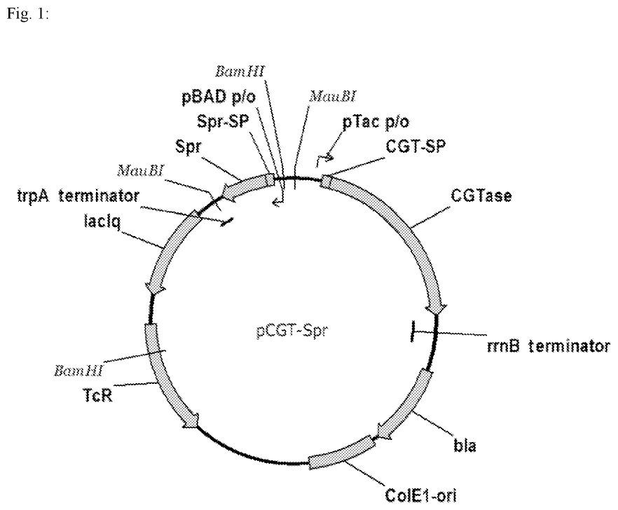

FIG. 1 shows the plasmid map of the plasmid pCGT-Spr.

FIG. 2 shows the plasmid map of the plasmid pCGT-YdhO.

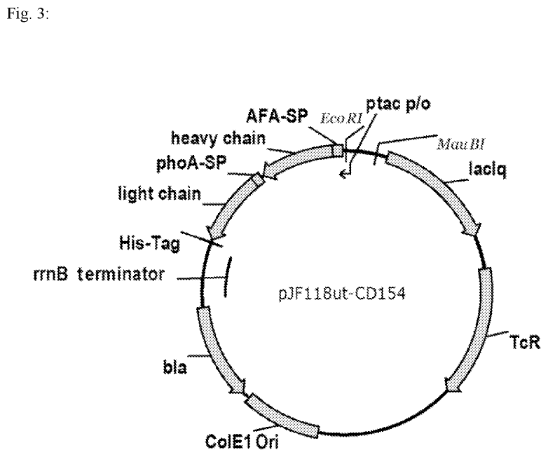

FIG. 3 shows the plasmid map of the plasmid pJF118ut-CD154.

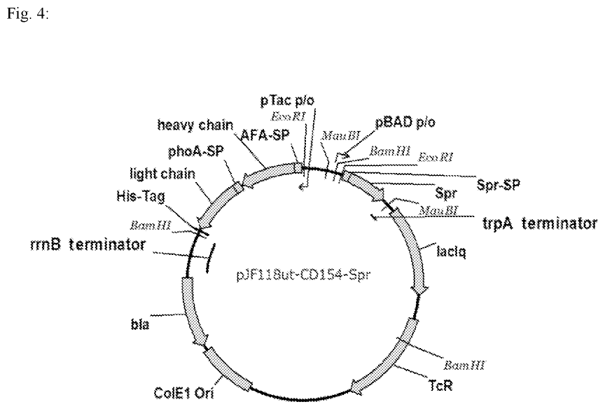

FIG. 4 shows the plasmid map of the plasmid pJF118ut-CD154-Spr.

FIG. 5 shows the analysis of the culture supernatants by SDS-PAGE, as described in Example 1. The samples are designated as follows: S: SeeBlue Prestained Protein Standard, 1: Supernatant from W3110/pCGT after 65 h, 2: Supernatant from W3110/pCGT-Spr with no addition of arabinose after 65 h, 3: Supernatant from W3110/pCGT-Spr with addition of arabinose after 65 h.

The abbreviations used in the figures represent DNA regions encoding the following functions:
tac p/o: tac promoter/operator
pBAD p/o: arabinose promoter/operator
bla: β-lactamase gene (ampicillin resistance)
TcR: tetracycline resistance
lacIq: repressor of the tac promoter
cgt-SP: signal peptide of CGTase
CGTase: cyclodextrin glycosyltransferase
ColE1: origin of replication
phoA-SP: phoA signal peptide
AFA-SP: derivative of the signal peptide of CGTase
rrnB terminator: terminator region of the rrnB gene
trpA terminator: terminator region of the trpA gene
Spr: Spr peptidoglycan peptidase
Spr-SP: signal peptide of Spr
YdhO: YdhO peptidoglycan peptidase
HisTag: histidine tag
light chain: antibody fragment comprising the domains VL and CL
heavy chain: antibody fragment comprising the domains VH and CH1
BamHI/MauBI/EcoRI: restriction sites of the corresponding restriction endonucleases

### EXAMPLES

The invention is described in more detail hereinbelow with reference to exemplary embodiments, without being limited thereby.

All the molecular-biology methods used, such as polymerase chain reaction (PCR), gene synthesis, isolation and purification of DNA, modification of DNA by restriction enzymes and ligase, transformation, etc., were carried out in the manner known to a person skilled in the art, described in the literature or recommended by the respective manufacturers.

### Description of the Plasmids:

pCGT:

The production of the plasmid pCGT is described in Example 4 of US 2008/0254511 A1, and the plasmid map is specified in FIG. 4 of US 2008/0254511 A1.

Essentially, the plasmid contains not only the gene for resistance to tetracycline, but also, inter alia, the structural gene of the cyclodextrin glycosyltransferase (CGTase) from Klebsiella pneumoniae M5al including the native CGTase signal sequence. The expression of the CGTase gene is under the control of the tac promoter.

pCGT-Spr:

To obtain pCGT-Spr (see FIG. 1 for the plasmid map), a DNA fragment was produced by means of gene synthesis by Eurofins Genomics. Said DNA fragment xI (specified in SEQ ID No. 1) contained:
nucleotides 1136-1304 from GenBank entry X81837.1 containing the arabinose promoter (pBAD promoter) and also the operators O1 and I2+I1 and the CAP binding site (nucleotides 10-178 of SEQ ID No. 1),
the Shine-Dalgarno sequence (nucleotides 203-208 of SEQ ID No. 1) and
a nucleotide fragment encoding a fusion of
i the Spr protein from E. coli K12 (nucleotides 216-782 of SEQ ID No. 1) and
ii the terminator of the trpA gene from E. coli (nucleotides 812-837 of SEQ ID No. 1).

Said DNA fragment xI was cut using the restriction enzyme MauBI and ligated with the expression vector pCGT, which had been cut using the same restriction enzyme. The cloning was done in an undirected manner; however, work was done with the plasmid in which the DNA fragment was inserted in the same reading direction as the gene encoding the CGTase, with verification carried out via the restriction pattern of the restriction enzyme BamHI and sequencing. Said plasmid was referred to as pCGT-Spr and encodes the Spr protein.

pCGT-YdhO:

To obtain pCGT-YdhO (see FIG. 2 for the plasmid map), a DNA fragment was produced by means of gene synthesis by Eurofins Genomics. Said DNA fragment xII (specified in SEQ ID No. 2) contained:
nucleotides 1136-1304 from GenBank entry X81837.1 containing the arabinose promoter (pBAD promoter) and also the operators O1 and I2+I1 and the CAP binding site (nucleotides 10-178 of SEQ ID No. 2),
the Shine-Dalgarno sequence (nucleotides 203-208 of SEQ ID No. 2) and
a nucleotide fragment encoding a fusion of
i the YdhO protein from E. coli K12 (nucleotides 216-1028 of SEQ ID No. 2) and
ii the terminator of the trpA gene from E. coli (nucleotides 1064-1089 of SEQ ID No. 2).

Said DNA fragment xII was cut using the restriction enzyme MauBI and ligated with the expression vector pCGT, which had been cut using the same restriction enzyme. The cloning was done in an undirected manner; however, work was done with the plasmid in which the DNA fragment was inserted in the same reading direction as the gene encoding the CGTase, with verification carried out via the restriction pattern of the restriction enzyme BamHI and sequencing. Said plasmid was referred to as pCGT-YdhO and encodes the YdhO protein.

pJF118ut-CD154:

The plasmid pJF118ut described in US 2008/076157 A was used as the starting vector for the cloning and expression of the genes of the Fab fragment of the humanized monoclonal anti-CD154 antibody 5c8, the sequence of which is published in Karpusas et al. 2001 (Structure 9, pages 321-329). pJF118ut is a derivative of the known expression vector pKK223-3 (Amersham Pharmacia Biotech) and deposited at the DSMZ-German Collection of Microorganisms and Cell Cultures GmbH (Braunschweig) under the number DSM 18596.

To obtain pJF118ut-CD154 (see FIG. 3 for the plasmid map), a DNA fragment was produced by means of gene synthesis by eurofins Genomics. Said DNA fragment xIII (specified in SEQ ID No. 3) comprised a fusion consisting of:
i the signal sequence disclosed in US 2008/076157 under SEQ ID No. 2 and derived from the signal sequence of a CGTase from Klebsiella pneumoniae M5a1 (nucleotides 25-114 of SEQ ID No. 3) and
ii the reading frame for the heavy chain (VH-CHI domains) of the Fab fragment of the humanized monoclonal anti-CD154 antibody 5c8, encoding amino acids 1-221 of the sequence published in Karpusas et al. 2001 in FIG. 3 (nucleotides 115-777 of SEQ ID No. 3),
iii the phoA signal sequence (nucleotides 800-862 of SEQ ID No. 3),
iv the reading frame for the light chain (VL-CL domains) of the Fab fragment of the humanized monoclonal anti-CD154 antibody 5c8, as published by Karpusas et al. 2001 in FIG. 3 (nucleotides 863-1516 of SEQ ID No. 3) and
v nucleotides 1517-1546 of SEQ ID No. 3, encoding a linker of 4 amino acids in length and a hexahistidine tag.

Said DNA fragment xIII was cut using the restriction enzymes EcoRI and PdmI and ligated with the expression vector pJF118ut, which had been cut using EcoRI and SmaI. The resulting plasmid, in which the expression of the genes for the heavy and light chain of the Fab fragment was under the control of the tac promoter, was designated pJF118ut-CD154.

pJF118ut-CD154-Spr:

The DNA fragment xI encoding Spr under the control of the arabinose promoter and flanked by the trpA terminator, as described above for pCGT-Spr, was inserted into the plasmid pJF118ut-CD154 via the MauBI restriction site. The cloning was done in an undirected manner; however, work was done with the plasmid in which the DNA fragment xI was inserted in the opposite reading direction to the DNA fragment xIII encoding CD154, with verification carried out via the restriction pattern of the restriction enzyme BamHI and sequencing. Said plasmid was referred to as pJF118ut-CD154-Spr (see FIG. 4 for the plasmid map).

### Example 1: Fermentative Production of Cyclodextrin Glycosyltransferase (CGTase) in a Stirred Tank Fermenter

For the production of the cyclodextrin glycosyltransferase (CGTase) from Klebsiella pneumoniae M5a1, the E. coli strain W3110 (ATCC 27325) was transformed with the plasmids pCGT, pCGT-Spr and pCGT-YdhO by common methods (e.g., TSS transformation). The selection for plasmid-containing cells was done by means of tetracycline (20 mg/l). The E. coli strains were designated W3110/pCGT, W3110/pCGT-Spr and W3110/pCGT-YdhO.

a)

CGTase production was carried out in stirred tank fermenters.

The fermenter filled with 1.2 l of the fermentation medium (1.5 g/l KH₂PO₄: 5 g/l (NH₄)₂SO₄: 0.5 g/l MgSO₄×7 H₂O: 0.225 g/l CaCl₂×2H₂O, 0.075 g/l FeSO₄×7 H₂O: 1 g/l Na₃ citrate×2H₂O: 0.5 g/l NaCl: 1 ml/l trace element solution (0.15 g/l Na₂MoO₄×2H₂O; 2.5 g/l Na₃BO₃; 0.7 g/l CoCl₂×6H2O; 0.25 g/l CuSO₄×5 H₂O; 1.6 g/l MnCl₂×4 H₂O; 0.3 g/l ZnSO₄×7H₂O); 5 mg/l vitamin B1; 3 g/l phytone peptone (BD 211906): 1.5 g/l yeast extract (Oxoid LP0021): 10 g/l glucose: 20 mg/l tetracycline) was inoculated to 0.1 OD₆₀₀ with a preliminary culture of W3110/pCGT or W3110/pCGT-Spr which had been cultured in LB medium (5 g/l yeast extract (Oxoid LP0021), 10 g/l tryptone (Oxoid LP0042), 5 g/l NaCl), additionally containing 1 ml/l trace element solution (0.15 g/l Na2MoO4×2H2O; 2.5 g/l Na3BO3; 0.7 g/l CoCl2×6H2O; 0.25 g/l CuSO4×5H2O; 1.6 g/l MnCl2×4H2O; 0.3 g/l ZnSO4×7H2O), 3 g/l glucose, 0.55 g/l CaCl2 and 20 mg/l tetracycline, in a shake flask for 7 h. The fermentation was thereby started (time point 0, start of fermentation). During the fermentation, a temperature of 30° C. was set and the pH was kept constant at a value of 7.0 by metering in NH₄+OH or H₃PO₄. Glucose was metered in over the fermentation, with a glucose concentration of <5 g/l being striven for. The expression of the CGTase was induced by addition of isopropyl β-D-thiogalactopyranoside (IPTG) to 0.15 mM after 21 h (at the end of the logarithmic growth phase). For the production of Spr, use was made of the basal expression of the arabinose promoter, which leads to the formation of low amounts of the Spr protein in the course of fermentation. The fact that the arabinose promoter allows low production of the target protein even in the absence of inducer has been described in the prior art (Malachowska and Olszewski, Microb Cell Fact (2018) 17:40).

Before inoculation, the medium in the fermenter was stirred at 400 rpm and sparged with 1.67 vvm (volume of air per volume of culture medium per minute) of compressed air sterilized via a sterile filter. Under these starting conditions, the optical oxygen sensor (VisiFerm DO225, Hamilton) was calibrated to 100% saturation before the inoculation. The target value for the O₂ saturation during the fermentation was set to 30% of this value. The O₂ saturation was measured via the oxygen sensor during the fermentation and captured by the fermenter control DCU (digital control unit, Sartorius Stedim). After the O₂ saturation fell below the target value, the stirring speed was increased continuously under software control to a maximum of 1500 rpm in order to bring the O₂ saturation back to the target value.

After 65 h of fermentation, samples were collected, the cells were removed from the fermentation medium by centrifugation, and the CGTase content in the fermentation supernatant was determined by the following enzyme assay:
Assay buffer: 5 mM Tris HCl buffer, 5 mM CaCl₂)×2H2O, pH 6.5
Substrate solution: 10% starch solution (Merck No. 1.01252) in assay buffer, pH 6.5
Assay mix: 0.2 ml of substrate solution+0.2 ml of centrifuged (5 min, 12 000 rpm) culture supernatant
Reaction temperature: 40° C.

### Enzyme Assay:

Preadjusting the temperature of substrate solution and centrifuged culture supernatant (approx. 5 min at 40° C.)
Preparing the assay mix by rapid mixing (whirl mixer) of substrate solution and centrifuged culture supernatant, the centrifuged culture supernatant being diluted with assay buffer if necessary so that a value of 0.9-1.5 g/l CD is determined in the subsequent HPLC analysis:
Incubating for 3 min at 40° C.
Stopping the enzyme reaction by addition of 0.6 ml of methanol and rapid mixing (whirl mixer)
Cooling the mix on ice (approx. 5 min)
Centrifuging (5 min, 12 000 rpm) and pipetting off the clear supernatant
Analyzing the amount of CD produced by means of HPLC: The analysis was carried out on an Agilent HP 1100 HPLC system with a Nucleodur 100-3 NH₂-RP column (150 mm×4.6 mm, Macherey-Nagel) and 64% acetonitrile in water (v/v) as mobile phase, at a flow rate of 2.1 ml/min. Detection was achieved via an RI detector (1260 Infinity RI, Agilent) and quantification was performed on the basis of the peak area and an α-CD standard (Cavamax W6-8 Pharma, Wacker Chemie AG).

Calculation of enzyme activity: A=G*V1*V2/(t*MG) [U/ml]
A=activity,
G=CD content in mg/l
V1=dilution factor in the assay mix
V2=dilution factor of the culture supernatant before use in the assay:
   if undiluted, then: V2=1
t=reaction time in min
MG=molecular weight in g/mol (MG_{CD}=973 g/mol)
1 unit (U)≙1 μmol/l product (CD)/min

Table 1 shows the respectively achieved cyclodextrin glycosyltransferase yields.

TABLE 1
CGTase yields in the fermentation supernatant
after 65 h of culturing.

| Strain | CGTase (U/ml) |
|---|---|
| W3110/pCGT | 31 |
| W3110/pCGT-Spr | 295 |

To examine whether the release of CGTase by production of Spr is attributable to increased cell lysis, the cell dry weight and the live cell count (LCC) of the cultures was determined at the end of the fermentation. To determine the live cell count, samples of the cultures were diluted 108-fold with LB medium in a final volume of 1 ml, and 100 μl of the dilution were plated out in each case on LB agar plates containing 20 mg/l tetracycline. The colonies grown were counted and, taking into account the dilution factor, the live cell count of the starting cultures was calculated at 50-109 for W3110/pCGT and at 39.109 for W3110/pCGT-Spr. To determine the cell dry weight, samples containing 1 ml of fermenter culture were transferred into reaction vessels, the empty weight of which had been determined beforehand. After centrifugation (5 min, 12 000 rpm), the supernatants were removed and the cell pellets were dried in an incubator (>48 h at 60° C.). Thereafter, the vessels containing the dried cell pellet were weighed out and the cell dry weight (CDW) was calculated from the difference between the weight of the vessels containing dried cell pellet and the weight of the empty vessels. The cell dry weights were 45 g/l for W3110/pCGT and 55 g/l for W3110/pCGT-Spr.

Strong cell lysis leads to the release of host cell protein. This was ruled out by analysis of the protein content of the culture supernatants by means of SDS-PAGE (FIG. 5, samples 1 and 2). After 65 h of fermentation, samples were collected and the cells were removed from the fermentation medium by centrifugation. The cell-free supernatant was diluted 1:10 with water, of which 4 μl was admixed with 4 μl of 2×Tris-Glycine SDS Sample Buffer (Invitrogen, Carlsbad, USA, cat. No. LC2676) containing 1 ml/l NuPAGE Antioxidant (Invitrogen, Carlsbad, USA, cat. No. NP0005) and 5% dithioerythritol and boiled at 99° C. in a thermal block for 5 min. The sample was centrifuged at 10 000 rpm for 20 s and 2 μl was loaded in each case onto a 12% NuPAGE Bis-Tris Gel (1.0 mm×15 wells, Invitrogen, Carlsbad, USA, cat. No. NP0343) and the proteins were resolved by electrophoresis in 1×MOPS buffer (NuPAGE MOPS SDS Running Buffer (20×), Invitrogen, Carlsbad, USA, cat. No. NP0001; diluted with water to 1×) at 95 V for 3 h. The standard loaded was See Blue Prestained (Invitrogen, Carlsbad, USA: cat. No. 100006636). The proteins were stained with Instant Blue (Coomassie-based staining solution for protein gels, Expedeon Ltd., Cambridge, UK, cat. No. ISB1L) at room temperature for 1 h and then destained in water overnight at room temperature. Besides the band for the CGTase protein, only minor contamination with other proteins can be identified in the gel. In particular, no increased release of the overproduced Spr protein (18 kDa) can be seen.

b)

Moreover, the production of the CGTase was carried out with the strains W3110/pCGT-Spr and W3110/pCGT-YdhO as described above. The production of YdhO was induced 48 h after the start of fermentation by switching the pure glucose feed to a constant feed of glucose/arabinose mix of 3 g/1*h in the glucose: arabinose ratio of 10:1. The production of Spr was induced 59 h after the start of fermentation by a single addition of arabinose to the culture such that a final concentration of 1 g/l was achieved. Harvesting was carried out 65 h after the start of fermentation. Table 2 shows the CGTase yields thus achieved.

TABLE 2
CGTase yields in the fermentation supernatant
after 65 h of culturing.

| Strain | CGTase (U/ml) |
|---|---|
| W3110/pCGT-Spr | 1130 |
| W3110/pCGT-YdhO | 1187 |

As described above, the cell dry weight was determined after 65 h of culturing. What were obtained were values of 50 g/l for W3110/pCGT-Spr and 41 g/l for W3110/pCGT-YdhO.

The culture supernatant from W3110/pCGT-Spr was analyzed by SDS-PAGE and showed that, besides the release of CGTase, there was no appreciable release of E. coli-endogenous proteins (FIG. 5, sample 3).

### Example 2: Fermentative Production of a Fab Antibody Fragment

For the production of the CD154 Fab fragment, the E. coli strain W3110 was transformed with the plasmids pJF118ut-CD154 and pJF118ut-CD154-Spr by common methods (e.g., TSS transformation). The selection for plasmid-containing cells was done by means of tetracycline (20 mg/l). The E. coli strains were designated W3110/pJF118ut-CD154 and W3110/pJF118ut-CD154-Spr. Said E. coli strains were fermented in stirred tank fermenters, as described for the fermentation of the E. coli pCGT strains in Example 1. In contrast to Example 1, CD154 Fab production was induced by 0.15 mM IPTG 26 h after the start of fermentation and Spr production was induced by switching the pure glucose feed to a constant feed of glucose-arabinose mix of 3 g/1*h in the glucose:arabinose ratio of 2:1 after 41 h.

After 48 h, samples were collected, the cells were removed from the culture medium by centrifugation, and the supernatant was analyzed to determine the CD154 Fab fragment released into the culture medium.

The CD154 Fab fragment was quantified via a sandwich ELISA assay known to a person skilled in the art. This involved using an immobilized anti-human IgG (Fd) antibody (The Binding Site, product No. PC075) as catcher and a peroxidase-conjugated goat anti-human kappa light chain antibody (Sigma, product no. A 7164) as detection antibody. Quantification was achieved by conversion of the chromogenic substrate Dako TMB+ (Dako #S1599) by the peroxidase and the associated absorption change at 450 nm. The ELISA was calibrated using the Fab fragment “Human Fab/Kappa” (Bethyl Laboratories, product number: P80-115).

TABLE 3
Yields of CD154 antibody fragment in
the culture supernatant after 48 h.

| | CD154 antibody fragment (mg/l) in the |
|---|---|
| Strain | supernatant |
| W3110/pJF118ut-CD154 | 5 |
| W3110/pJF118ut-CD154-Spr | 1045 |

To examine the influence of the expression of Spr on cell growth. the cell dry weight of the cultures was determined as described in Example 1 at the end of the fermentation after 48 h. The results are combined in Table 4.

TABLE 4
Cell dry weights (CDW) of the CD154-producing
strains in the fermenter after 48 h.

| Strain | CDW (g/1) |
|---|---|
| W3110/pJF118ut-CD154 | 48 |
| W3110/pJF118ut-CD154-Spr | 43 |

1-15. (canceled)

16. A method for fermentative production of recombinant proteins, characterized in that a bacterial strain containing an open reading frame encoding a signal peptide and a recombinant protein under the control of a functional promoter, contains an additional open reading frame encoding a signal peptide and a peptidoglycan peptidase under the control of a functional promoter, wherein the peptidoglycan peptidase is (i) Spr, (ii) YdhO or (iii) Spr and YdhO,
is cultured in a fermentation medium, the fermentation medium is removed from the cells after the fermentation and the recombinant protein is isolated from the fermentation medium,
wherein the recombinant protein is released from the periplasm of the cell and the yield of recombinant protein which is released into the culture medium is at least 1.1 times as high as the yield which can be achieved with corresponding bacterial strains without overproduction of the peptidoglycan peptidase.

17. The method for fermentative production of recombinant proteins as claimed in claim 16, characterized in that the bacterial strain is Gram-negative bacteria.

18. The method for fermentative production of recombinant proteins as claimed in claim 16, wherein the bacterial strain is a strain of the species Escherichia coli.

19. The method for fermentative production of recombinant proteins as claimed in claim 16, wherein Spr is the sequence specified by amino acids 27-188 in SEQ ID No. 5.

20. The method for fermentative production of recombinant proteins as claimed in claim 16, wherein YdhO is the sequence specified by amino acids 28-271 in SEQ ID No. 7.

21. The method for fermentative production of recombinant proteins as claimed in claim 16, wherein the functional promoter which controls the expression of the open reading frame encoding the peptidoglycan peptidase is an inducible promoter.

22. The method for fermentative production of recombinant proteins as claimed in claim 16, wherein the recombinant protein is a heterologous protein.

23. The method as claimed in claim 16, wherein the expression of the peptidoglycan peptidase is induced.

24. The method as claimed in claim 16, wherein the expression of the peptidoglycan peptidase is induced after the induction of the expression of the recombinant protein.

25. The method as claimed in claim 16, wherein, 5 to 24 hours after induction of the expression of the additional peptidoglycan peptidase, the cell dry weight after the removal of the fermentation medium differs by not more than 20% from the cell dry weight of a cell culture at the same time point from a fermentation method for a bacterial strain which does not express the additional peptidoglycan peptidase.
